(19) United States
(12) Patent Application Publication
(10) Pub. No.: US 2023/0133317 A1
(43) Pub. Date: May 4, 2023

(54) Taste Masked And Rapidly Disintegrating Ultra Thin Iron Orodispersible Film And A Process Thereof

(71) Applicant: Aavishkar Oral Strips Private Limited, Hyderabad (IN)

(72) Inventors: Dinesh Reddy APPENAHALLI RAVICHANDRA, Hyderabad (IN); Anubhab MUKHERJEE, Hyderabad (IN); Naresh Babu ATCHUTA VENKATA, (US)

(21) Appl. No.: 17/913,469

(22) PCT Filed: Mar. 22, 2021
(86) PCT No.: PCT/IN2021/050296

§ 371 Date: Sep. 22, 2022

(30) Foreign Application Priority Data
Mar. 23, 2020 (IN) 202041012621

(51) Int. Cl. A61K 33/26; A61K 47/38; A61K 47/40; A61K 9/00; A61K 9/50

(52) U.S. Cl. CPC A61K 33/26; A61K 47/38; A61K 47/40; A61K 9/006; A61K 9/501

(57) ABSTRACT

Taste masked and rapidly disintegrating ultra thin iron orodispersible film and a process thereof The present disclosure generally relates to self administrable oral formulation of 5 iron. Specifically, the disclosure provides ultra thin orodispersible films (ODFs)—a novel patient centered innovation and excellent alternatives to traditional solid dosage forms—tablets and capsules. The instant disclosure provides iron containing ODFs that are taste masked and disintegrate rapidly when administered to tongue. They are easy to carry and will be used by subjects such as paediatrics, 10 geriatrics and pregnant women and many others to treat iron deficiency and its related disorders. These iron ODFs are a potential alternative, particularly, to subjects who depend a lot on iron supplements infused via parenteral route.

### TECHNICAL FIELD

The present disclosure is in relation to oral formulation of ‘Iron’. Particularly, the disclosure provides Orodispersible Film (ODF) formulation of iron and a method to prepare the same.

### BACKGROUND

Deficiency of iron is the most common micronutrient deficiency that affects almost 24% of the world's population. Iron has several vital functions in the human body. For instance, it helps in carrying oxygen from lungs to tissues, participates as a co-factor of essential enzymatic reactions in neurotransmission, synthesis of steroid hormones, synthesis of bile salts, and detoxification processes in the liver. Deficiency of iron could lead to anaemia. In addition, it's deficiency also leads to increase in maternal & foetal mortality, increased risk of premature delivery with low birth weight, learning disabilities (dyslexia) and delayed psychomotor development, reduced work capacity, impaired immunity (prone to infections), and inability to maintain body temperature.

Iron is administered in various forms for the treatment of iron deficiency and also as a prophylactic to supply the minimum daily recommended allowance. A variety of iron compounds have been administered in the past—including but not limiting to ferric and ferrous forms of elemental iron as salts, complexes, hydrates, chelates. Presently available oral iron preparations suffer with various disadvantages such as low bioavailability and/or substantial side effects. Several attempts were made in the past to create pharmaceutical iron dosage forms that not only provide sufficient iron for absorption to treat deficiencies but also could overcome its side effects. Primarily, the side effects of orally administered iron formulations are due to its large doses required to facilitate required absorption. As a result, the presence of unabsorbed iron tends to remain in the gastrointestinal tract causing irritation, abdominal pain, heartburn, constipation, diarrhoea, nausea, and vomiting. Alternately, Iron therapy could be carried out by parenteral route of administration (either intravenously (IV) or intramuscularly (IM)). Nonetheless, parenteral therapy is also associated with substantial side effects, including anaphylactic shock, injection site issues, hypotension, muscle cramps, dizziness, headache, graft complications, hypertension, chest pain, ear pain, and peripheral oedema. In addition to this, it is also associated with high cost and discomfort associated with injections and thus resulting in poor patient compliance. Therefore, there is a need for rapid release self administrable oral iron formulations that get absorbed faster resulting in higher bioavailability and thereby eliminating the side effects associated with present oral and parenteral delivery formulations of iron. Accordingly, the present disclosure is directed to overcome one or more limitations stated above or any other limitation associated with the prior arts.

### SUMMARY

Accordingly, the present disclosure provides a taste masked and rapidly disintegrating ultra thin iron orodispersible film composition comprising of microencapsulated iron; beta cyclodextrin; flavouring agent; and calcium carboxy methyl cellulose, wherein microencapsulated iron to beta cyclodextrin ratio is 1:0.543, microencapsulated iron to flavoring agent, preferably kiwi flavor ratio is 1:0.326 and microencapsulated iron to calcium carboxy methyl cellulose ratio is 1:4 along with pharmaceutically acceptable excipients; and is also disclosed is a process for preparing taste masked and rapidly disintegrating ultra thin orodispersible film formulation comprising of microencapsulated iron at a concentration of 37% w/w, pullulan at a concentration ranging from 44% to 47% w/w, beta cyclodextrin at a concentration of 20% w/w, mannitol and calcium carboxy methyl cellulose each at a concentration of 5% w/w and sweetening agent at a concentration ranging from 0.8% to 5% w/w, polyethylene glycol at a concentration of 2% w/w, plasticizer at a concentration ranging from 2% to 4% w/w, lecithin at a concentration ranging from 2% to 4% w/w, malic acid at a concentration of 4% w/w, ascorbic acid at a concentration of 0.1% w/w and kiwi flavor at a concentration ranging from 8% to 12% w/w, comprising steps of preparing microencapsulated iron capsules having iron at a concentration ranging from 36% to 40%; dissolving pullulan in water by continuous stirring and allowing it to stay overnight to obtain clear viscous slurry; mixing microencapsulated iron obtained in above step with beta cyclodextrin in water under continuous stirring followed by addition of plasticizers, sweetening agents, sialagogues, disintegrating agents, ascorbic acid, lecithin solution and kiwi flavor under continuous stirring for a time period ranging from 15 to 30 minutes; combining the slurry obtained in the previous step with clear pullulan slurry to obtain homogeneous and uniform casting slurry followed by deaeration under vacuum to remove air bubbles; and casting the deaerated slurry over a layering machine followed by drying and cutting to obtain ultra thin iron orodispersible films.

It is to be understood that the aspects and embodiments of the disclosure described above may be used in any combination with each other. Several of the aspects and embodiments may be combined together to form a further embodiment of the disclosure. The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF ACCOMPANYING DRAWINGS

The features of the present disclosure will become more fully apparent from the following description and appended claims, taken in conjunction with the accompanying drawings. Understanding that these drawings depict only several embodiments in accordance with the disclosure and are therefore not to be considered limiting of its scope the disclosure will be described with additional specificity and detail through use of the accompanying drawings.

FIG. 1: shows HPLC chromatograms (a) Blank; (b) Standard (c) Sample ODF films of iron.

The FIGURE depicts embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

Before explaining any one embodiment of the present disclosure by way of drawings, experimentation, results, and pertinent procedures, it is to be understood that the disclosure is not limited in its application to the details as explained in below embodiments set forth in the following description or illustrated in the drawings, experimentation and/or results. The disclosure is further capable of other embodiments which can be practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

### Definitions

The terminology used in the description of the invention herein is for the purpose of describing particular embodiments only and is not intended to be limiting of the invention.

As per the European Pharmacopoeia (Ph. Eur.), Orodispersible Films (ODFs) is a type of oromucosal preparation which is defined as “single or multilayered sheets of suitable materials, to be placed in the mouth where they disperse rapidly”. On the contrary, United States Pharmacopoeia (USP) employs a different terminology and called them as ‘Oral Films’ and defined as “Thin sheets that are placed in the oral cavity. They contain one or more layers. A layer might or might not contain API”. Alternately, United States Food and Drug Administration's structured product labelling term (SPLT) termed ODFs as ‘Soluble Films’ having code C42984 and defined as “A film that will dissolve in a liquid solvent to form a solution”. Clinical Data Interchange Standards Consortium (CDISC) defined them as “A thin layer or coating which is susceptible to being dissolved when in contact with a liquid”.

“Active agent,” as used herein, refers to a compound or molecule that has a therapeutic, prophylactic, or nutritive effect when delivered to a subject.

The word ‘Iron’ in the present disclosure refers to an “iron compound,” as used herein, refers to a complex comprising elemental iron and an additional atom, ion, or molecule, and includes iron salts, iron chelates, iron complexes, and polymer-bound iron. Similarly, an “iron complex,” refers to elemental iron in neutral or cationic form covalently or electro statically linked to an additional atom, ion, or molecule. In addition, it also refers to “iron chelate,” refers to an iron cation and anions that surround the iron cation and are joined to it by electrostatic bonds. It can also refer to an encapsulated form of iron.

A “disease or disorder characterized by an iron deficiency,” as used herein, refers to any disease or disorder in which whole body stores of iron are less than desired. Low body stores may be indicated by various symptoms including a blood level of iron that is below normal. The normal serum iron level for human adults is considered to be ranging between 60 to 170 μg/dL. The protein ‘Ferritin’ helps store iron in the human body, and a low level of ferritin indicates low iron levels. In addition, lower haemoglobin levels also indicate deficiency of iron. The normal blood haemoglobin levels in men ranges from 13.5 to 17.5 grams per decilitre and in women it ranges from 12.0 to 15.5 grams per decilitre. Further, Red Blood Cells play a role in supplying oxygen to different parts of the body via blood flow through the circulatory system. Deficiency in iron makes the red blood cells smaller and paler in colour than normal. The percentage of blood volume made by the red blood cells is estimated as ‘Haematocrit’, whose normal values range from 35.5 to 44.9 for women and 38.3 to 48.6 for adult men. Nonetheless, these values do change with age and gender.

As it is known, anaemia is an indication with scarcity in the population of red blood cells (RBCs) or mal-functional RBCs in the body. This results in reduced oxygen flow to the body's organs as the consequence of iron deficiency (lack of iron unit of haemoglobin present in RBC which is the carrier of oxygen). Medical symptoms include fatigue, skin pallor, light-headedness, shortness of breath and dizziness or a fast heartbeat. Globally, many of the pregnant women suffer from this. It turns out that healthy kidneys produce a hormone called erythropoietin (EPO) which triggers the bone marrow to produce RBC. Diseased or damaged kidneys (for instance, in case of Chronic Kidney Disease (CKD)) refuse to produce substantial EPO to prompt bone marrow to generate lesser RBC, which causes anaemia. The prevalence of anaemia increases with the deterioration of renal function, subjecting the patients for the two forms of dialysis, namely, haemodialysis and peritoneal dialysis. The major treatment modalities involve administration of oral or intravenous iron and erythropoietin (EPO). Many studies demonstrated that raising the low levels of iron via oral administration with 15-20% intestinal absorption has healing effect on anaemic patients in conservative phase albeit it shows gastrointestinal side-effects. On the other hand, haemodialysis patients prefer intravenous route which has severe side effects like allergy, systemic inflammation, etc.

An oral liposomal iron, ferric pyrophosphate encapsulated within a phospholipids membrane, tends to have a lower occurrence of gastrointestinal side effects, without increasing the inflammation of the patient. Herein, the present disclosure provides a novel oral iron delivery system which can potentially replace the intravenous dosing and open a new door for future studies to combat anaemia.

The present disclosure is in relation to a taste masked and rapidly disintegrating ultra thin iron orodispersible film composition comprising microencapsulated iron; beta cyclodextrin; flavouring agent; and calcium carboxy methyl cellulose, wherein microencapsulated iron to beta cyclodextrin ratio is 1:0.543, microencapsulated iron to flavoring agent, preferably kiwi flavor ratio is 1:0.326 and microencapsulated iron to calcium carboxy methyl cellulose ratio is 1:4 along with pharmaceutically acceptable excipients.

In another embodiment of the present disclosure, it provides microencapsulated iron having iron concentration ranging from 36% to 40% w/w, preferably 38% w/w.

In yet another embodiment of the present disclosure, it provides for ultra thin iron orodispersible film formulation comprising of microencapsulated iron at a concentration of 37% w/w, pullulan at a concentration ranging from 44% to 47% w/w, beta cyclodextrin at a concentration of 20% w/w, mannitol and calcium carboxy methyl cellulose each at a concentration of 5% w/w and sweetening agents at a concentration ranging from 0.8% to 5% w/w, polyethylene glycol at a concentration of 2% w/w, plasticizer at a concentration ranging from 2% to 4% w/w, lecithin at a concentration ranging from 2% to 4% w/w, malic acid at a concentration of 4% w/w, ascorbic acid at a concentration of 0.1% w/w and kiwi flavor at a concentration ranging from 8% to 12% w/w.

In still another embodiment of the present disclosure, the sweetening agents are selected from a group comprising of glucose, fructose and steviose glycosides.

In still another embodiment of the present disclosure, the plasticizer is selected from a group comprising of sorbitol, surfactants, glycerol and glycerol oleate.

The present disclosure is in relation to a process for preparing taste masked and rapidly disintegrating ultra thin orodispersible film formulation comprising of microencapsulated iron at a concentration of 37% w/w, pullulan at a concentration ranging from 44% to 47% w/w, beta cyclodextrin at a concentration of 20% w/w, mannitol and calcium carboxy methyl cellulose each at a concentration of 5% w/w and sweetening agents at a concentration ranging from 0.8% to 5% w/w, polyethylene glycol at a concentration of 2% w/w, plasticizer at a concentration ranging from 2% to 4% w/w, lecithin at a concentration ranging from 2% to 4% w/w, malic acid at a concentration of 4% w/w, ascorbic acid at a concentration of 0.1% w/w and kiwi flavor at a concentration ranging from 8% to 12% w/w, comprising steps of: preparing microencapsulated iron capsules having iron at a concentration ranging from 36% to 40%; dissolving pullulan in water by continuous stirring and allowing it to stay overnight to obtain clear viscous slurry; mixing microencapsulated iron obtained in first step with beta cyclodextrin in water under continuous stirring followed by addition of plasticizers, sweetening agents, sialagogues, disintegrating agents, ascorbic acid, lecithin solution and kiwi flavor under continuous stirring for a time period ranging from 15 to 30 minutes; combining the solution obtained in the above step with clear pullulan slurry to obtain homogeneous and clear casting slurry/dispersion followed by deaeration under vacuum to remove air bubbles; and casting the deaerated casting dispersion over a layering machine followed by drying and cutting to obtain ultra thin iron orodispersible films.

In another embodiment of the present disclosure, it provides a process for microencapsulation of iron, comprising steps of adding iron salts to a solution of sodium alginate to obtain a homogeneous mixture followed by drop wise addition to a solution of calcium chloride or calcium acetate to obtain microcapsules of iron; filtering the microcapsules of iron by vacuum filtration; and washing the microcapsules of iron with water to remove soluble iron salts followed by filtration to obtain reddish brown microencapsulated iron particles free of soluble iron and covered with calcium layer.

In yet another embodiment of the present disclosure, drying of films is carried out at a temperature of about 60° C. and the disintegration time is less than 30 seconds.

In still another embodiment of the present disclosure, ascorbic acid helps in iron absorption and is anti-oxidant.

In still yet another embodiment of the present disclosure, mannitol is used to prevent caking of pullulan slurry and aids in smooth peeling of the film from the film forming machine slab.

Additionally, the disclosure is further illustrated by the following examples, which are not to be construed in any way as imposing limitations upon the scope of the present invention. On the contrary, it is to be clearly understood that various other embodiments, modifications, and equivalents thereof, after reading the description herein in conjunction with the drawings and appended claims, may suggest themselves to those skilled in the art without departing from the spirit and scope of the presently disclosed and claimed invention.

### Example 1: Process for Preparing Microencapsulated Iron

To an aqueous solution of sodium alginate, approximately 8.2 gm of ferric saccharate or ferric chloride or ferrous sulphate heptahydrate (36% to 40% of iron or Fe) was added and stirred until it dissolves to obtain a homogeneous mixture. Microencapsulation of iron was performed by drop wise addition of the mixture to a solution of calcium chloride, wherein the molar concentration of calcium chloride solution was ranging from 0.1 to 1.0 M. As an alternate, solutions of calcium acetate with suitable molar concentration can also be employed in the process instead of calcium chloride. The formed microcapsules were separated by a simple vacuum filtration technique. Soluble iron salts are removed by suspending the microcapsules in plain distilled water, this step is repeated until the microcapsules are free from soluble iron salts. Finally, the microcapsules were subjected for vacuum filtration to obtain microencapsulated iron particles that are free from soluble iron slats. Yields ranging from about 25 to 30 gm of wet iron microcapsules were obtained, which were reddish brown in colour. Nonetheless, the color of microcapsules was varying depending upon the type of iron salt employed in the process. With the above process and the order of addition of ingredients it helps in obtaining microcapsules of iron, wherein iron is at the core that is surrounded by calcium layer. In short, the prepared microcapsules have inner iron rich core surrounded by calcium rich outer layer.

Using the above method, different concentrations of microencapsulated iron are prepared. For our study, we have prepared encapsulated iron concentration ranging from 36% to 40% iron and preferably 38% iron was employed in preparing orodispersible films of iron. The concentration of iron, alginate and calcium salts is also determined by various instrumental methods of analysis. By the above process the taste of iron is masked as it is released slowly from the matrix.

### Example 2: Characterization of Microencapsulated Iron

The particle size of microencapsulated iron or iron capsules is determined by optical microscopy. The size of the capsules was ranging from about 5 to 20 μm. As regards the concentration of iron and calcium, they were quantified by spectroscopic method known as Inductively Coupled Plasma-optical Emission Spectroscopy (ICP-OES). The prepared capsules were also subjected to stability studies, as per ICH guidelines, to understand its release profile during storage. The release of both iron and calcium was used to indicate the capsule stability. Less the iron released, better the stability of the capsules as the iron is intact without exposing to the outside environment. Hardest stability conditions (high temperature 37° C. and presence of water) lead to release of negligible amount of iron (<1.0%). All in all, the stability data clearly indicated that the encapsulated iron is stable.

### Example 3: Method to Prepare Microencapsulated Iron Orodispersible Films

(ODFs): The list of ingredients employed and its role or use are listed out in the below table 1.

TABLE 1
List of ingredients and its role

| Sl. | Name of the | |
|---|---|---|
| No. | Ingredient | Role/Use of the ingredient |
| 1. | Microencapsulated | Active nutrient |
| | iron | |
| 2. | Pullulan | Film forming polymer |
| 3. | Beta cyclodextrin | Complexing agent |
| 4. | Zinc lactate | Taste masking agent |
| 5. | Dicalcium Phosphate | Diluent |
| 6. | Mannitol | Anti-caking agent and also facilitates |
| | | smooth peeling of the film from the |
| | | slab of layering machine. |
| 7. | Glucose | Sweetening agent |
| 8. | Fructose | Sweetening agent |
| 9. | Steviose 100 | Sweetening agent |
| 10. | Stevirome 5000 | Sweetening agent |
| 11. | Steviol glycosides | Sweetening agent |
| 12. | Calcium carboxy | Disintegrating agent |
| | methyl cellulose | |
| 13. | PEG 600 | Plasticizer and wetting agent |
| 14. | Sorbitol 70% | Plasticizer |
| 15. | Glycerol Triacetate | Plasticizer |
| 16. | Tween 80 | Surfactant - reduces surface tension |
| | | between the oily flavoring agents with |
| | | aqueous portion of the formulation. |
| 17. | Lecithin | Emulsifying agent |
| 18. | Ascorbic acid | Promotes iron absorption and also acts as |
| | | anti-oxidant. |
| 19. | Malic acid | Sialagogue - saliva stimulating agent |
| 20. | Glycerol | Plasticizer |
| 21. | Glycerol oleate | Plasticizer |
| 22. | Grape flavour | Flavoring agent |
| 23. | Kiwi flavor | Flavoring agent |
| 24. | Water | Solvent |

The film forming material, pullulan, is dissolved in water by stirring and left it overnight to obtain clear viscous slurry, termed as polymer solution. Lecithin was also dissolved in a separate portion of the solvent. Microencapsulated iron and beta cyclodextrin were mixed in water under continuous stirring followed by addition of mannitol, desired sweetening agent selected from a group comprising of steviosides, glucose and fructose. Other ingredients, such as calcium carboxyl methyl cellulose, ascorbic and malic acid were also added and continued to stir for about 10 minutes. Thereafter, the stirring is still continued for another 5 minutes by adding polyethylene glycol, sorbitol and kiwi flavor. The solution of microencapsulated iron along with all other excipients and the lecithin solution are added to the polymer solution under continuous stirring for a time period of about 10 minutes. Mixing under continuous stirring is carried out till homogenous and clear slurry is obtained. Thereafter, the final clear slurry is subjected for de-aeration under vacuum (pressure between 600 to 700 mm of Hg) to remove air bubbles, if any, for a time period ranging from 2 to 3 hours. After successfully removing the air bubbles, the casting solution is layered over a layering machine with predetermined parameters on thickness (250 μm) and other parameters (RPM 2.0 to 3.0). Once the layering is completed, the thin film is slit and cut using the machine to a dimension of 32×25 mm. The prepared films are subjected to drying at a temperature of about 60° C. Thereafter, the films are packed in aluminium foils and stored in a dessicator to prevent any atmospheric moisture or microorganism attack for further characterization.

Using the above general method different batches of ODFs are prepared and evaluated/characterized. Different batches of iron ODFs are depicted in below table 2 along with concentration of each ingredient employed. There were a total of six batches that were prepared by trial and error method.

TABLE 2
Different batches of microencapsulated iron ODFs

| | Different batches with concentration of | | | | | |
|---|---|---|---|---|---|---|
| List of | both actives and excipients (% w/w) | | | | | |
| ingredients | F1 | F2 | F3 | F4 | F5 | F6 |
| Micro- | 37 | 37 | 37 | 37 | 37 | 37 |
| encapsulated | | | | | | |
| iron | | | | | | |
| Pullulan | 43 | 47 | 47 | 47 | 47 | 44 |
| Beta cyclodextrin | — | — | — | — | 20 | 20 |
| Zinc lactate | — | 5 | — | — | — | — |
| DCP (Dicalcium | 5 | 5 | — | — | — | — |
| Phosphate) | | | | | | |
| Mannitol | 5 | 5 | 5 | 5 | 5 | 5 |
| Glucose | 5 | 5 | 5 | 5 | 5 | 5 |
| Fructose | 5 | 0.8 | 5 | 5 | 5 | 5 |
| Steviose 100 | — | — | — | — | — | 0.8 |
| Stevirome 5000 | — | — | — | — | — | 1.8 |
| Steviol glycosides | 0.8 | 5 | 0.8 | 0.8 | 1.2 | — |
| Calcium CMC | 5 | 1 | 5 | 5 | 5 | 5 |
| PEG 600 | — | — | — | — | — | 2 |
| Sorbitol 70% | — | — | — | — | — | 4 |
| Glycerol | — | 1 | 1 | 1 | 2 | — |
| Triacetate | | | | | | |
| Tween 80 | 1 | — | — | — | — | — |
| Lecithin | 1.8 | 1 | 1.8 | 1.8 | 2 | 4 |
| ascorbic acid | 0.15 | 1 | 0.1 | 0.1 | 0.1 | 0.1 |
| Malic acid | — | 4 | 4 | 4 | 4 | 4 |
| Glycerol | 5 | 5 | 5 | 5 | 4 | — |
| Glycerol oleate | 0.13 | 0.13 | 0.13 | 0.13 | — | — |
| Grape flavour | 6 | 8 | 8 | 8 | — | — |
| Kiwi flavor | — | — | — | 12 | 8 | 12 |
| Water (ml) | 175 | 200 | 200 | 200 | 200 | 200 |
| Total weight in | 120 | 131 | 125 | 137 | 145 | 150 |
| mg/unit ODF | | | | | | |

The microencapsulated iron prepared as per example 1 attempt's to mask the taste of iron only to an extent. Most importantly, the microencapsulated iron cannot be directly administered to patients/subjects. It needs to be converted into a suitable pharmaceutical formulation using pharmaceutically acceptable additives. Such pharmaceutical formulations can be readily administered to subjects/patients who are in need thereof. Therefore, converting them into readily administrable orodispersible film (ODFs) with rapid disintegration and taste masking potential are the key acceptable characteristics for the success of delivering iron via the oral route instead of delivering it by using traditional solid dosage forms—tablets. Different batches (F1 to F6) were formulated with an ultimate goal of obtaining a microencapsulated iron ODF that has very low disintegration time and excellent taste masking potential to gain acceptability by the subjects and thereby the compliance.

First and foremost, the microencapsulated iron obtained and characterized under example numbers 1 and 2 are used in formulating ODFs. The percentage of iron in the microencapsulated iron was ranging from 36% to 40%, preferably 38% was used.

Turning now towards the taste masking agent ‘Cyclodextrins (CDs)’, they are cyclic oligosaccharides with a hydrophilic outer surface and a lipophilic central cavity. On account of their relatively hydrophobic interiors, CDs have the ability to form inclusion complexes with a wide range of drug substances. The ability of CDs to form inclusion complexes is explored in the present disclosure to mask the bitter taste of iron. Beta CDs are used in the present disclosure to mask the bitter taste of iron. The complex formed between iron and beta CDs is having high stability/binding constant. This helps in preventing the iron release per se in the oral cavity. In the present disclosure, the ODFs undergo rapid disintegration to release the microencapsulated iron which the subject swallows along with the saliva and the iron is released in the stomach of the subject. In addition to beta CDs, zinc lactate—another taste masking agent was tried for few of the batches of the microencapsulated iron ODFs in a ratio of (microencapsulated iron:zinc lactate) 1:0.136, and the percentage was 3.817%. Nonetheless, ODFs prepared using zinc lactate (Formulation F2) showed low level of acceptance by the subjects. In addition, the disintegration time was higher >1 minute. To fix the disintegration issue, calcium carboxy methyl cellulose was employed at a slightly higher concentration that what was employed in formulation F2. This helped in significantly decreasing the disintegration time, which is <30 seconds. The ratio of microencapsulated iron vis-à-vis the disintegrating agent (calcium carboxy methyl cellulose) was 1:4. Nonetheless, formulation F3 also suffered with lack of acceptable taste (bitterly tasting ODF) but the same was showing disintegration time of less than 30 seconds.

Further, beta CDs were employed in formulating formulations F5 and F6. The beta CDs and microencapsulated iron ratio was (microencapsulated iron:beta cyclodextrin) 1:0.543. In addition, to beta CDs, formulation F5 and F6 were employed with unique flavour ratio of (microencapsulated iron: kiwi flavour) 1:0.326. The flavor of preference was kiwi flavor. Both formulations F5 and F6 exhibited excellent taste masking potential and rapid disintegration time, which is less than 30 seconds.

### Example 4: Physical Methods for Characterization of Microencapsulated Iron ODFs

(a) Visual inspection: Visual inspection was carried out from sampled ODFs. The colour of the films was reddish brown color and free from air bubbles.

(b) Shape: The ODFs could be cut into desired shape. For instance, rectangular shaped ODFs were cut using the cutting machine of size ranging 4 cm² to 6 cm². These sizes of ODFs are highly comfortable for self-administration by patients/subjects across all the age groups.

(c) Thickness: Thickness of the ODF is measured using a micrometer (digital) which was found to be ranging from 0.110 to 0.125 Mm.

(d) Average weight: ODFs having an area of 700 mm² were weighed using an electronic balance. The average weight obtained is a mean weight variation of the film. This gives a general confirmation of the fact that both the drug and excipients are uniformly distributed in the ODF and one has obtained an ODF weighing about 150 mg.

(e) Folding endurance (FE): This test is performed manually. The ODF of the uniform cross-sectional area is folded repeatedly until it breaks. FE value is the number of times the sample ODF is folded repeatedly without cracking. High FE value is a direct indication to establish the fact that ODF is indeed associated with higher mechanical strength. The FE value for the iron ODFs was ranging from 15 to 20.

### Example 5: In-Vitro Methods for Characterization of Microencapsulated Iron ODFs

(a) Disintegration Test: Disintegration of ODF is critical quality attribute that helps in gaining patient compliance. The ODFs are expected to rapidly disintegrate when administered to tongue. There exist several methods for determining the disintegration time of an ODF. Most popular methods are petri dish method, slide frame method, drop method, hollow glass cylinder method, slide frame and ball method and others. The most popular LDR-LED sensing method can also be utilized for predicting both the start time and end disintegration time of an ODF. In the present disclosure, PharmaTest®—ODF disintegration tester was employed to study the disintegration time of the microencapsulated iron ODF. Standard procedure was followed in testing the disintegration time using disintegration medium—‘phosphate buffer’ having pH 6.8. It was observed that the disintegration time of all the films was less than 30 seconds, except the films of formulation F2.

In addition, petri dish method was also used in studying the disintegration time of microencapsulated ODFs. The Petri dish method is much simpler compared to the other methods as it just involves placing a film of size 2×2 cm in a petri dish with 10 mL of water followed by recording the time required for the complete disintegration of the film. In order to simulate the movement of tongue, ‘orbital bath shaker’ was used by maintaining the speed of about 50 rpm at a temperature of 37° C. The disintegration time of all the ODFs (except F2) was found to be less than 30 seconds.

(b) Estimation of moisture content by Karl Fischer (KF) titration method: This method helps in determining even the lowest amount of water content in any ODF sample. It employs methanol or anhydrous dimethyl sulfoxide as a solvent. The selected solvent determines the solubility of an ODF for the analysis. In the present method, suitable amount of ODF sample, say 500 mg of ODF sample is transferred into titration vessel and the titration was continued till the electrometric end point. Every time, before adding the sample, titrate the vessels content to electrometric end point to neutralize the moisture interference during the process.

The moisture content was determined using the below formula:

$$\frac{Volume of KF reagent ( mL ) ⨯ KF factor}{Weight of sample ( mg ) ⨯ 1000} ⨯ 100$$

The water content in microencapsulated iron ODF samples was found to be between 6.0 to 8.0% w/w.

(c) Iron identification by HPLC: The retention time of the peak of the test solution correspond to the chromatogram of the standard solution. The chromatographic conditions, mobile phase, blank, sample and standard solution preparations are explained in the below section.

(d) Assay by HPLC Method:

The chromatographic conditions are listed out below:

| Parameter | Details | |
|---|---|---|
| Column size | C18, 250 mm × 4.6 mm; | |
| | 5 μm or Equivalent column | |
| Column make | Baker bond | |
| Mobile phase flow rate | 1.0 | ml/min |
| Std/Sample injection volume | 20 | μl |
| Temperature | 25° C. ± 0.5° C. | |
| Detection wavelength | 210 | nm |
| Run time | 15 | minutes |

Preparation of Phosphate Buffer pH 2.5: Accurately weighed 2.72 g of Potassium dihydrogen orthophosphate (KH₂PO₄) was transferred into beaker containing 1000 ml of Milli Q water mixed well and adjusted the pH to 2.5±0.5 using ortho phosphoric acid.

Preparation of Mobile Phase: Measure 970 mL of Phosphate Buffer having pH of 2.5 and add 30 mL of methanol. Mix well and filter through 0.45 μm millipore filter and sonicate for 10 minutes.

Preparation of Diluent. Measure and transfer 12.7 ml of concentrated HCl into 500 ml volumetric flask containing 250 ml of water. Add 25 mL of ortho phosphoric acid mix well and make up the volume to 500 mL using water.

Preparation of standard solution: Accurately weigh and transfer 35 mg of iron into a 100 mL volumetric flask. Add 50 mL of diluent followed by sonication for 2 minutes and make up to volume using diluent. Mix well and transfer the solution to centrifuge tube and centrifuge for 5 minutes at 5000 RPM and take the supernatant solution for the analysis.

Preparation of Sample: Weigh and transfer 10 microencapsulated iron ODF samples into 100 mL volumetric flask, add 50 mL of diluent and sonicate for 30 minutes. Dissolve the samples by cyclic mixing until completely dissolved and make up the volume using diluent and filter using whatman filter paper No. 1.

Procedure: Separately inject 20 μL of blank, standard solution and sample solution into the chromatography system, record the chromatograph by maintaining the chromatographic conditions identified in the above table and the measure the response for the major peaks.

System suitability parameters: The relative standard deviation for replicate standard injections is not more than 2.0%. The tailing factor is not more than 2.0. Theoretical plates should be not less than 2000. Inject the solution as per the sequence of injection given below.

| Sequence of injections: | No. of Injection |
|---|---|
| Blank | 01 |
| Standard Solution | 05 |
| Sample Solution | 02 |
| Bracketing Standard | 01 |

Calculation:

$$\frac{Sample Avg area}{Std Avg area} ⨯ \frac{Std weight}{100} ⨯ \frac{100}{Sample weight} ⨯ \frac{Average weight}{Label claim} ⨯ WS Purity$$

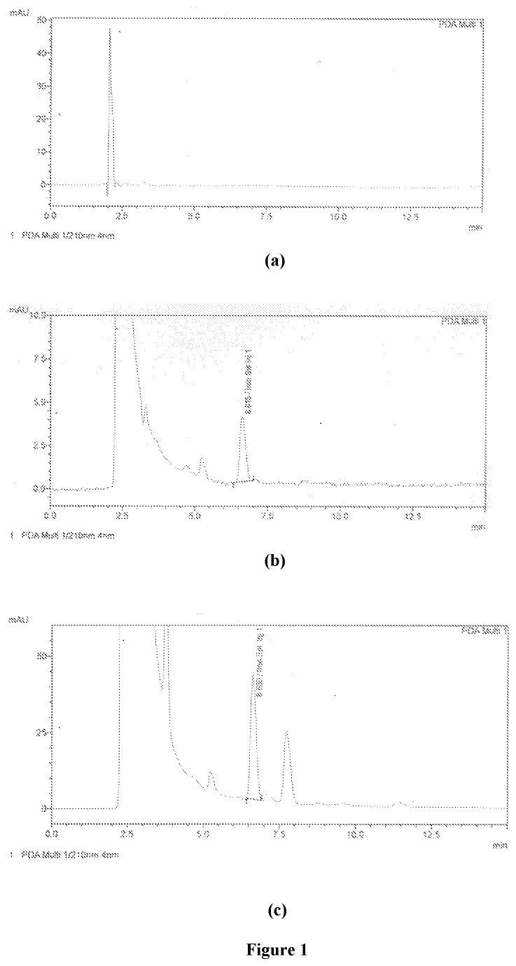

The iron content in the sample ODF was found to be 99.4% of label claim as average value for the optimized formulation which explained by chromatograms obtained by HPLC as shown in FIG. 1.

### Example 6: In-Vivo Methods for Characterization of Microencapsulated Iron ODFs

In order to determine the patient/subject acceptability of microencapsulated iron ODFs, taste and palatability are crucial factors that need to be determined. Under in vitro conditions, biochemical, biomimetic or ion selective detectors are utilized. There has recently been an increasing use of special panels dedicated to taste evaluation—“electronic tongues” (multi-sensor taste detectors with pattern recognition systems) which seem to be good alternatives to pre-testing of the formulation. Taste masking properties can also be evaluated in vitro using a dissolution test. The most reliable, but ethically problematic, is the in vivo test in human volunteers. Before the examination, subjects evaluate their sensory sensibility thresholds for respective tastes, using four standard substances: tartaric acid (sour), sucrose (sweet), sodium chloride (salty), quinine (bitter). It is proposed to conduct the study in the following stages: rinsing the mouth with distilled water, placing the required amount of active and then a film sample with the same active content on the tongue for 30 seconds, spitting the drug and rinsing the mouth with water. For taste evaluation, the scale with the following values is usually utilized: 0—free of bitter taste, 1—slightly bitter, 2—moderately bitter, 3—very bitter and 4—extremely bitter to taste. The scores given by volunteers are tabulated in table 3. From the below table, it is abundantly evident that formulations F5 and F6 are free of bitter taste, with the exception of two volunteers who rated the taste as slightly bitter.

TABLE 3
Volunteer taste scores

| | Volunteers | | | | | | | | | |
|---|---|---|---|---|---|---|---|---|---|---|
| Formulation | 1 | 2 | 3 | 4 | 5 | 6 | 7 | 8 | 9 | 10 |
| F1 | 1 | 2 | 2 | 1 | 2 | 3 | 1 | 3 | 3 | 2 |
| F2 | 3 | 3 | 2 | 3 | 2 | 3 | 2 | 3 | 1 | 1 |
| F3 | 2 | 1 | 2 | 1 | 2 | 2 | 1 | 2 | 3 | 0 |
| F4 | 1 | 1 | 2 | 2 | 1 | 2 | 2 | 2 | 0 | 1 |
| F5 | 0 | 0 | 0 | 0 | 0 | 1 | 0 | 0 | 0 | 0 |
| F6 | 0 | 0 | 0 | 0 | 0 | 1 | 0 | 0 | 0 | 0 |

### Example 7: Other Characterization Tests

(a) Pesticide residue testing for Characterization of Microencapsulated Iron ODFs: The microencapsulated iron ODF samples were tested for presence of pesticides by in-house standard testing procedure. The test procedure involves testing the samples by gas chromatography-mass spectroscopy (GC-MS) method. Almost 144 pesticides were found to be well below the quantification limits (0.01 mg/Kg).

(b) Test for heavy metals: The microencapsulated iron ODF samples were tested for presence of heavy metals by in-house standard testing procedures. The test procedure involves testing the samples by ‘Inductively Coupled Plasma Mass Spectrometry (ICP-MS) for presence of arsenic, cadmium, mercury and lead. All the tested heavy metals were found to less than 0.05 to 0.1 ppm or mg/Kg.

(c) Microorganism: The microencapsulated iron ODF samples were tested for presence of microorganisms by in-house standard testing procedures. The test procedure involves testing the samples for presence of microorganisms namely yeast and moulds, E. coli (bacteria), Salmonella, Staphylococcus aureus and Pseudomonas aeruginosa. Except yeast and moulds (with <10 CFU/g), THE remaining microorganism were absent.

Advantages:
Microencapsulated iron ODFs of the present disclosure are the most powerful and transformational alternative to the extensively used solid and parenteral dosage forms (tablets, capsules, intravenous and intramuscular injections). These iron ODFs are easy to carry and are cost effective.
The iron ODFs of the present disclosure are rapid-release and self-administrable oral iron formulations that get absorbed faster resulting in higher bioavailability of iron.
The iron ODFs of the present disclosure can be used in situations where iron is required as a supplement to pregnant women. These iron ODFs are very helpful for pregnant women who are depending on parenteral route and are living in remote villages.
ODFs of the present disclosure are excellent in gaining patient compliance in general and particularly in patients with dysphagia (difficulty in swallowing), Parkinson's disease, mucositis and vomiting tendency.
The beauty of the ODFs of the present disclosure is that they are unique active iron delivery systems that don't need water for consumption by the subjects/patients. Thus, they are definitely of great advantage for third world countries that don't always have clean drinking water readily available to consume medication.
Method of synthesis of ODFs is simple yet elegant and they can be easily scaled up in industry.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1) A taste masked and rapidly disintegrating ultra-thin iron orodispersible film composition comprising: (a) microencapsulated iron; (b) beta cyclodextrin; (c) flavouring agent; and (d) calcium carboxy methyl cellulose, wherein the microencapsulated iron to beta cyclodextrin ratio is 1:0.543, microencapsulated iron to flavoring agent, preferably kiwi flavor ratio is 1:0.326 and microencapsulated iron to calcium carboxy methyl cellulose ratio is 1:4 along with pharmaceutically compatible excipients.

2) The composition as claimed in claim 1, wherein microencapsulated iron having iron concentration ranging from 36% to 40%, preferably 38%.

3) The composition as claimed in claim 1, wherein said ultra-thin iron orodispersible film is a pharmaceutical formulation comprising of microencapsulated iron at a concentration of 37% w/w, pullulan at a concentration ranging from 44% to 47% w/w, beta cyclodextrin at a concentration of 20% w/w, mannitol and calcium carboxy methyl cellulose each at a concentration of 5% w/w, sweetening agent at a concentration ranging from 0.8% to 5% w/w, polyethylene glycol at a concentration of 2% w/w, plasticizer at a concentration ranging from 2% to 4% w/w, lecithin at a concentration ranging from 2% to 4% w/w, malic acid at a concentration of 4% w/w, ascorbic acid at a concentration of 0.1% w/w and kiwi flavor at a concentration ranging from 8% to 12% w/w.

4) The composition as claimed in claim 3, wherein said sweetening agents are selected from a group comprising of glucose, fructose and steviose glycosides.

5) The composition as claimed in claim 3, wherein said plasticizer is selected from a group comprising of sorbitol, glycerol triacetate, polyethylene glycol, glycerol and glycerol oleate.

6) A process for preparing taste masked and rapidly disintegrating ultra thin orodispersible film formulation as claimed in claim 1, comprising steps of:
a) preparing microencapsulated iron capsules having iron at a concentration ranging from 36% to 40%;
b) dissolving pullulan in water by continuous stirring and allowing it to stay overnight to obtain clear viscous slurry;
c) mixing microencapsulated iron obtained in step (a) with beta cyclodextrin in water under continuous stirring followed by addition of plasticizers, sweetening agents, sialagogues, disintegrating agents, ascorbic acid, lecithin solution and kiwi flavor under continuous stirring for a time period ranging from 15 to 30 minutes;
d) combining the solution obtained in step (c) with clear pullulan slurry obtained in step (b) to obtain homogeneous and clear casting slurry followed by deaeration under vacuum to remove air bubbles; and
e) casting the deaerated casting slurry obtained in step (d) over a layering machine followed by drying and cutting to obtain ultra-thin iron containing orodispersible films.

7) The process as claimed in claim 6, wherein said microencapsulated iron preparation comprises steps of:
a) adding iron salts to a solution of sodium alginate to obtain a homogeneous mixture followed by drop wise addition to a solution of calcium chloride or calcium acetate to obtain microcapsules of iron;
b) filtering the microcapsules of iron obtained in step (a) by vacuum filtration; and
c) washing the microcapsules of iron obtained in step (b) with water to remove soluble iron salts followed by filtration to obtain reddish brown microencapsulated iron particles free of soluble iron and covered with calcium layer.

8) The process as claimed in claim 6, wherein drying of films is carried out at a temperature of about 60° C. and the disintegration time is less than 30 seconds.

9) The process as claimed in claim 6, wherein ascorbic acid helps in iron absorption and is anti-oxidant.

10) The process as claimed in claim 6, wherein mannitol is used to prevent caking of pullulan slurry and aids in smooth peeling of the film from the film forming machine slab.
